(19) United States
(12) Patent Application Publication
(10) Pub. No.: US 2011/0092592 A1
(43) Pub. Date: Apr. 21, 2011

(54) DIAGNOSIS AND TREATMENT OF HEPATIC DISORDER

(76) Inventor: Takashi Yano, Shizuoka (JP)

(21) Appl. No.: 12/997,446

(22) PCT Filed: Jun. 12, 2009
(86) PCT No.: PCT/JP2009/060797

§ 371 Date: Dec. 10, 2010

(30) Foreign Application Priority Data
Jun. 13, 2008 (JP) 2008 156036

(51) Int. Cl. A61K 31/232; A61K 31/202; A61K 31/201; A61K 31/231; A61P 1/16

(52) U.S. Cl. 514/549; 514/560

(57) ABSTRACT

Disclosed is a diagnosis or treatment method which utilizes the content of a specific fatty acid in plasma as a marker that reflects the condition of NASH or NAFLD, or utilizes the above-mentioned content in combination with another test, another marker or the like.

### TECHNICAL FIELD

The present invention relates to methods of preventing/ameliorating or treating non-alcoholic fatty liver disease, non-alcoholic steatohepatitis in particular, and pharmaceutical compositions used in such methods.

### BACKGROUND ART

A group of liver diseases occurring in those having no alcohol drinking histories, including such hepatic disorders as simple fatty liver, steatohepatitis, fibrosis and cirrhosis, are collectively defined as non-alcoholic fatty liver disease (hereafter referred to as “NAFLD”) except for viral liver diseases, autoimmune liver diseases, and metabolic liver diseases such as hemochromatosis and Wilson's disease.

NAFLD can be divided on the basis of liver biopsy (pathological findings) into two stages, namely, simple fatty liver thought generally to be of good prognosis and non-alcoholic steatohepatitis (hereafter referred to as “NASH”) of bad prognosis, the latter being regarded as a severer form of NAFLD. Pathologic conditions determined by liver biopsy to be NASH, such as inflammation, pimelosis, fibrosis or cirrhosis, and liver cancer, are not different from those otherwise caused, and many of the hepatitides which are not considered as an alcoholic hepatic disorder, viral hepatitis or drug-induced hepatic disorder are expected to be a pathologic condition belonging to NASH (see Non-Patent Literature 1).

It is said that 20% of the population suffers from NAFLD, and 3% from NASH, in the United States. Also in Japan, such diseases are relatively often encountered upon a general medical practice, with the frequency of NAFLD in examinees being 8%, and it is estimated that the frequency of NASH in adult Japanese is at least 0.5 to 1%. Based on the fact that 13 million male and 10 million female adult Japanese have BMIs of 25 or greater indicating their obesity, domestic NAFLD patients are estimated to be 5 to 6 million in number and NASH patients approximately 300 to 500 thousand. In addition, lipid metabolism abnormality, hypertension, hyperglycemia and metabolic syndrome (hereafter referred to as “MetS”), all as defined in the criteria for diagnosis of MetS, have incidences as a complication of NAFLD of about 50%, about 30%, about 30% and about 40%, respectively (see Non-Patent Literature 1), so that it is expected that NASH will increase in number of cases and spread through younger generations along with the increase in lifestyle disease cases in the future. Moreover, a clinical problem is offered by a partial progress of hepatitis to cirrhosis, or even to liver cancer by the activation of stellate cells.

In “NASH•NAFLD no Shinryo Gaido (Guidelines for Diagnosis and Treatment of NASH and NAFLD)” of The Japan Society of Hepatology (Non-Patent Literature 1) reporting the effectiveness of the NASH treatment methods which have been attempted aiming at the amelioration of a variety of pathologic conditions, it is stated at the same time that no treatment method is established yet at present.

Specific examples of the treatment methods as described include methods using: insulin sensitizers, including biguanides (e.g., metformin), and thiazolidine derivatives (pioglitazone, rosiglitazone) as a PPAR-γ agonist; antioxidants such as vitamins, betaines (choline derivatives), and N-acetylcysteine; antihyperlipidemic agents, such as fibrate drugs (PPAR-α agonists), HMG-CoA reductase inhibitors (statins), and probucol; liver protection drugs such as ursodeoxycholic acid and polyene phosphatidylcholine (EPL); and angiotensin II receptor antagonists such as losartan.

There are reports on the administration of icosapentaenoic acid (hereafter referred to as “EPA”) or fish oil to NASH and NAFLD patients. For instance, it is reported that ω-3 polyunsaturated fatty acids (hereafter referred to as “PUFAs”), to be more specific, a mixture of EPA-E and ethyl docosahexaenate (hereafter referred to as “DHA-E”) can ameliorate hepatitis in patients with NAFLD (see Non-Patent Literature 2).

According to a latest report by Tanaka et al., amelioration of NASH is revealed by administering EPA-E of high purity at a dose of 2700 mg/day for 12 months, observing aspartate aminotransferase (hereafter referred to as “AST”) or alanine aminotransferase (hereafter referred to as “ALT”) enzyme, giving assessments by inflammatory cytokines or oxidative stress markers, and conducting liver biopsy after the period of administration and observation (see Non-Patent Literature 3).

It is proposed that ω-3 PUFAs be applied to the treatment of fatty liver in patients with NASH and so forth as a peroxisomal and/or mitochondrial β-oxidation stimulating agent (see Patent Literature 1).

With respect to the relationship between NASH and plasma fatty acids, it is described that 22 NASH patients and 6 healthy subjects were compared with one another in plasma total fatty acid level (fatty acid ester plus free fatty acid), and in free fatty acid level as well, in order to review the relationship between NASH and the accumulation of plasma fatty acids and, as a result, the NASH patients were found to have high saturated and monounsaturated fatty acid levels, with the palmitic acid (C16:0), palmitoleic acid (C16:1) and oleic acid (C18:1) levels being particularly high. It is also described that there were no significant differences between the NASH patients and the control group in the ratio C18:1/C18:0 or C20:4/C18:2 as a desaturase activity index (see Non-Patent Literature 4).

On the other hand, it is stated that ω-3 PUFAs reduced mature SREBP1c in the liver of the ob/ob mouse, which was obese and had fatty liver due to the deficiency of leptin, suppressed gene expression of fat synthesis-related enzymes such as fatty acid synthase (FAS) and stearoyl-CoA desaturase (SCD1), and reduced fatty acids, specifically palmitic acid (C16:0), palmitoleic acid (C16:1) and oleic acid (C18:1), in the liver (see Non-Patent Literature 5).

The “two-hit” theory positing about the mechanism of NASH onset that triglyceride (TG) deposition in hepatocytes (fatty liver) occurs initially (first hit), then some hepatocyte-damaging factor or other is added thereto (second hit) to result in the onset of NASH, is widely supported. SREBP1c is known as a protein promoting the transcription for fatty acid synthases and considered to be involved with the onset of fatty liver (first hit) (Non-Patent Literature 1), and it is known that ω-3 PUFAs reduce SREBP1c.

It, however, has not been investigated yet what relationship exists between the variation in a specified fatty acid composition ratio of the subject, who was diagnosed by liver biopsy as having NASH, that is to say, experienced the second hit, and to whom ω-3 PUFAs have been administered for a certain period of time, and the therapeutic effects on NASH. In other words, it is not known that the therapeutic effects of ω-3 PUFAs on NASH can be predicted or evaluated on the basis of the variation in a specified fatty acid composition ratio in a certain period of time.

At present, liver biopsy is indispensable to an affirmative diagnosis of NASH. Liver biopsy is also required for the determination of cured liver diseases because various markers used for such determination may not always reflect pathologic conditions faithfully. Liver biopsy, however, imposes a great burden to the patient's body, and to health care professional as well, so that a simple method for diagnosis of NASH or evaluation of a pathologic condition belonging to NASH is sought.

### CITATION LIST

### Patent Literature

Patent Literature 1: WO 2007/081773

### NON-PATENT LITERATURE

Non-Patent Literature 1: The Japan Society of Hepatology ed., “NASH•NAFLD no Shinryo Gaido (Guidelines for Diagnosis and Treatment of NASH and NAFLD),” BUNKODO CO., LTD., Aug. 22, 2006.
Non-Patent Literature 2: Alimentary Pharmacology & Therapeutics, Vol. 23, No. 8, pp. 1143-1151, Apr. 15, 2006.
Non-Patent Literature 3: Journal of Clinical Gastroenterology, Vol. 42, No. 4, pp. 413-418, 2008.
Non-Patent Literature 4: Clinical Nutrition 2002, 21(3), pp. 219-223.
Non-Patent Literature 5: Hepatology, Vol. 38, No. 6, pp. 1529-1539, 2003.

### SUMMARY OF INVENTION

### Technical Problems

It is an object of the present invention to provide an effective indexes reflecting a pathologic condition of a subject with NASH, and to provide a treatment method using such an index.

### Solution to Problems

The inventor of the present invention concentrated on researches in order to achieve the above objects, and found at last that a composition ratio of particular fatty acids in plasma serves as a good marker reflecting a pathologic condition of a subject with NASH, and that, in the subjects on whom prevention/amelioration or treatment of NAFLD, NASH in particular, with ω-3 PUFAs was performed, a combination of certain tests or markers is effective as an index to prophylactic/ameliorative or therapeutic effects. The present invention has been accomplished on the basis of such findings.

The present invention provides a method of treatment for NASH/NAFLD and/or a method of suppressing transition to cirrhosis/liver cancer, in which the effects of a therapeutic agent against NASH such as ω-3 PUFAs are determined by a periodical measurement of the index in question, and the subject can be treated while checking his/her responsiveness to the agent administered.

The present invention also provides a method for diagnosis of NASH using as an index a combination of composition ratios of particular fatty acids in the plasma or the liver, certain tests, or certain markers.

The present invention also provides a treatment method which starts administering a therapeutic agent against NASH such as ω-3 PUFAs to a subject if the subject has a high composition ratio of particular fatty acids in the plasma or the liver.

The present invention also provides a method of screening those subjects who are responsive to the administration of a therapeutic agent based on a composition ratio of particular fatty acids in the plasma or the liver.

The present invention also provides a method of administering a therapeutic agent against NASH such as ω-3 PUFAs while determining the effects thereof.

In other words, the present invention provides the following.

[1] A method of preventing/ameliorating or treating NASH or NAFLD in a subject, or a method of managing a subject, which comprises using a plasma, serum or liver fatty acid composition ratio of a subject as an index to evaluate the subject's condition or therapeutic effects.

[2] A method of preventing/ameliorating or treating non-alcoholic steatohepatitis, which comprises using one or more selected from the group consisting of the plasma oleic acid/stearic acid ratio, the plasma stearic acid/palmitic acid ratio and the plasma oleic acid/palmitic acid ratio of a subject as indices for the evaluation of the subject's condition or therapeutic effects, and administering to the subject a pharmaceutical composition containing as an active ingredient at least one selected from the group consisting of ω-3 polyunsaturated fatty acids as well as pharmaceutically acceptable salts and esters thereof.

[3] A method of preventing/ameliorating or treating non-alcoholic steatohepatitis, comprising the steps of:

(1) calculating one or more selected from among the plasma oleic acid/stearic acid ratio, the plasma stearic acid/palmitic acid ratio and the plasma oleic acid/palmitic acid ratio of a subject;
(2) administering to the subject for a certain period of time a pharmaceutical composition containing as an active ingredient at least one selected from the group consisting of ω-3 polyunsaturated fatty acids as well as pharmaceutically acceptable salts and esters thereof;
(3) calculating again one or more selected from among the plasma oleic acid/stearic acid ratio, the plasma stearic acid/palmitic acid ratio and the plasma oleic acid/palmitic acid ratio of the subject;
(4) comparing one or more selected from among the plasma oleic acid/stearic acid ratio, the plasma stearic acid/palmitic acid ratio and the plasma oleic acid/palmitic acid ratio that were calculated before administering the pharmaceutical composition with those calculated after administering the pharmaceutical composition so as to make an evaluation of the subject's condition or therapeutic effects;
(5) administering the pharmaceutical composition to the subject based on the evaluation; and
(6) thus preventing/ameliorating or treating non-alcoholic steatohepatitis.

[4] A method of preventing/ameliorating or treating non-alcoholic steatohepatitis in a subject suffering from non-alcoholic steatohepatitis, comprising the steps of:

(1) obtaining a first determination with respect to at least one out of the plasma oleic acid/stearic acid ratio, the plasma stearic acid/palmitic acid ratio and the plasma oleic acid/palmitic acid ratio of the subject;
(2) administering to the subject a pharmaceutical composition containing as an active ingredient at least one selected from the group consisting of ω-3 polyunsaturated fatty acids as well as pharmaceutically acceptable salts and esters thereof;
(3) obtaining a second determination with respect to at least one out of the plasma oleic acid/stearic acid ratio, the plasma stearic acid/palmitic acid ratio and the plasma oleic acid/palmitic acid ratio of the subject;
(4) making a comparison between the first and second determinations in order to evaluate the subject's condition;
(5) evaluating, based on the comparison between the first and second determinations, the treatment given to the subject in order to specify the appropriate therapeutic dosage of the pharmaceutical composition, the composition being suitable for the prevention/amelioration or treatment of non-alcoholic steatohepatitis; and
(6) thus preventing/ameliorating or treating non-alcoholic steatohepatitis.

[5] The method according to [4] as above, wherein the step of making a comparison between the first and second determinations further includes determining upon making the comparison whether or not at least one out of the plasma oleic acid/stearic acid ratio, the plasma stearic acid/palmitic acid ratio and the plasma oleic acid/palmitic acid ratio of the subject is reduced.

[6] The method according to [4] or [5] as above, wherein the pharmaceutical composition is administered for one month before the second determination is obtained.

[7] The treatment method according to any one of [1] through [6] as above, wherein the evaluation of the subject's condition or therapeutic effects is made using another test or marker in combination.

[8] The treatment method according to any one of [1] through [7] as above, wherein the prevention/amelioration or treatment method is continued for three months or longer.

[9] The treatment method according to any one of [1] through [8] as above, wherein the pharmaceutical composition is combined with a diet therapy.

[10] A pharmaceutical composition for use in the method of preventing/ameliorating or treating NASH or NAFLD, or the method of managing a subject, as above, which contains as an active ingredient at least one selected from the group consisting of ω-3 polyunsaturated fatty acids as well as pharmaceutically acceptable salts and esters thereof.

[11] A pharmaceutical composition for prevention/amelioration or treatment of non-alcoholic steatohepatitis, which contains as an active ingredient at least one selected from the group consisting of ω-3 polyunsaturated fatty acids as well as pharmaceutically acceptable salts and esters thereof, and which is administered by using values of one or more selected from the group consisting of the plasma oleic acid/stearic acid ratio, the plasma stearic acid/palmitic acid ratio and the plasma oleic acid/palmitic acid ratio of a subject as indices for the evaluation of the subject's condition or therapeutic effects.

[12] A pharmaceutical composition for prevention and/or treatment of non-alcoholic steatohepatitis, which is administered by:

(1) calculating values of one or more selected from the group consisting of the plasma oleic acid/stearic acid ratio, the plasma stearic acid/palmitic acid ratio and the plasma oleic acid/palmitic acid ratio of a subject;
(2) calculating values of one or more selected from the group consisting of the plasma oleic acid/stearic acid ratio, the plasma stearic acid/palmitic acid ratio and the plasma oleic acid/palmitic acid ratio of the subject to whom a pharmaceutical composition containing as an active ingredient at least one selected from the group consisting of ω-3 polyunsaturated fatty acids as well as pharmaceutically acceptable salts and esters thereof has been administered for a certain period of time after taking the plasma;
(3) comparing the values calculated in (1) with those calculated in (2) to make an evaluation of the subject's condition or therapeutic effects; and
(4) carrying out the administration based on the evaluation.

[13] A pharmaceutical composition containing as an active ingredient at least one selected from the group consisting of ω-3 polyunsaturated fatty acids as well as pharmaceutically acceptable salts and esters thereof, for reduction of values of one or more selected from the group consisting of the plasma oleic acid/stearic acid ratio, the plasma stearic acid/palmitic acid ratio and the plasma oleic acid/palmitic acid ratio of a patient with non-alcoholic steatohepatitis or NAFLD.

[14] A pharmaceutical composition for improvement of blood fatty acid composition of a patient with non-alcoholic steatohepatitis or NAFLD, which contains as an active ingredient at least one selected from the group consisting of ω-3 polyunsaturated fatty acids as well as pharmaceutically acceptable salts and esters thereof, and which reduces one or more selected from the group consisting of a plasma oleic acid/stearic acid ratio, a plasma stearic acid/palmitic acid ratio and a plasma oleic acid/palmitic acid ratio of a patient with non-alcoholic steatohepatitis or NAFLD.

[15] The pharmaceutical composition according to any one of [10] through [14] as above, wherein the plasma oleic acid/stearic acid ratio, the plasma stearic acid/palmitic acid ratio, and the plasma oleic acid/palmitic acid ratio are each a plasma free fatty acid composition ratio.

[16] Use of at least one selected from the group consisting of ω-3 polyunsaturated fatty acids as well as pharmaceutically acceptable salts and esters thereof for the manufacture of a medicament for use in the prevention/amelioration or treatment of NASH or NAFLD, or the management of a subject, using a plasma, serum or liver fatty acid composition ratio of a subject as an index for the evaluation of the subject's condition or therapeutic effects.

[17] Use of at least one selected from the group consisting of ω-3 polyunsaturated fatty acids as well as pharmaceutically acceptable salts and esters thereof for the manufacture of a medicament for use in the prevention/amelioration or treatment of NASH or NAFLD, or the management of a subject, using one or more selected from the group consisting of a plasma oleic acid/stearic acid ratio, a plasma stearic acid/palmitic acid ratio and a plasma oleic acid/palmitic acid ratio of a subject as indices for the evaluation of the subject's condition or therapeutic effects.

[18] At least one selected from the group consisting of ω-3 polyunsaturated fatty acids as well as pharmaceutically acceptable salts and esters thereof for use in the prevention/amelioration or treatment of NASH or NAFLD, or the management of a subject, using a plasma, serum or liver fatty acid composition ratio of a subject as an index for the evaluation of the subject's condition or therapeutic effects.

[19] At least one selected from the group consisting of ω-3 polyunsaturated fatty acids as well as pharmaceutically acceptable salts and esters thereof for use in the prevention/amelioration or treatment of NASH or NAFLD, or the management of a subject, using one or more selected from the group consisting of a plasma oleic acid/stearic acid ratio, a plasma stearic acid/palmitic acid ratio and a plasma oleic acid/palmitic acid ratio of a subject as indices for the evaluation of the subject's condition or therapeutic effects.

### ADVANTAGEOUS EFFECTS OF INVENTION

According to the present invention, the index for the evaluation of prophylactic/ameliorative or therapeutic effects on NASH is provided which enables a more effective prevention/amelioration or treatment of NASH. In addition, the subject in whom prophylactic/ameliorative or therapeutic effects on NAFLD or NASH are well gained and the subject in whom such effects are hardly gained can be distinguished from each other simply by using as an index a composition ratio of particular fatty acids in plasma or another test or marker. The present invention makes it possible to select a more effective treatment for the subject in whom less effects are gained by changing the dose or the treatment policy, and is thus clinically useful. The present invention, as facilitating the evaluation of therapeutic effects, allows a reduction in frequency of liver biopsy, burden on doctors and patients, or even in risk of medical mishaps.

Moreover, the subject's condition can be grasped, and it can be determined whether the treatment is effective at present or not, or is expected to become effective or not, by observing the fatty acid change in a relatively short term such as one to three months. An index provided by the present invention is useful because it reflects a pathologic condition of NASH prior to other test value or marker changes with NASH. Application of the inventive index to a treatment method can make treatment more appropriate to the subject.

### DESCRIPTION OF EMBODIMENTS

The present invention is detailed in the following.

In its first aspect, the present invention provides a method of preventing/ameliorating or treating NASH or NAFLD, or a method of managing a subject, which includes using a plasma, serum or liver fatty acid composition ratio of a subject as an index to evaluate the subject's condition or therapeutic effects.

The method is preferably a method of preventing/ameliorating or treating NASH or NAFLD, or a method of managing a subject, in which a pharmaceutical composition containing ω-3 PUFAs is administered to a subject using a plasma, serum or liver fatty acid composition ratio of the subject as an index.

While the fatty acid to be used for the index (marker) of the present invention is not particularly limited, it is preferable to use a fatty acid measurable by a known technique such as twenty-four fatty acid fractionation. Examples include myristic acid, palmitic acid, palmitoleic acid (16:1), stearic acid, and oleic acid, among which palmitic acid, stearic acid and oleic acid are preferable, with the most preferred being oleic acid. It is desirable that a fatty acid is determined in amount as the mole percent of the total amount of fatty acids. A particularly preferable index is the composition ratio between two or more fatty acids, such as the oleic acid (OA)/stearic acid (SA) ratio, the stearic acid (SA)/palmitic acid (PA) ratio, the oleic acid (OA)/palmitic acid (PA) ratio, the palmitoleic acid/palmitic acid ratio, the stearic acid/myristic acid ratio, and the palmitic acid/myristic acid ratio, with the OA/SA, SA/PA and OA/PA ratios being favorable. The above fatty acids may be used for the index in combination of three or four.

The twenty-four fatty acid fractionation is a testing technique for preparing fractions of certain 24 fatty acids, including both saturated and unsaturated ones, and quantifying the fractions by gas chromatography. To be more specific: Fatty acids in the plasma are extracted by, for instance, the method of Folch et al. (Folch, J. et al., J. Biol. Chem., 226, pp. 497-509, 1957). Each of the fatty acids is methylated with boron trifluoride and methanol using tricosanoic acid (C23:0) as an internal standard, and the methyl-esterified form of each fatty acid may be measured or quantified by using a gas chromatograph such as GC-17A (manufactured by SHIMADZU CORPORATION) and a capillary column such as BPX70 (0.25 mm ID×30 m; manufactured by SGE International Pty. Ltd.) in a non-limitative manner.

In the treatment method of the present invention, it is desirable that the evaluation of therapeutic effects, or of the severity of a disease, based on a plasma fatty acid composition ratio as an index, and the administration of a pharmaceutical composition are performed in parallel or repeated alternately.

More specifically, the method of the present invention is desirably a method of preventing/ameliorating or treating non-alcoholic steatohepatitis, in which:

(1) one or more selected from among the oleic acid/stearic acid ratio, the stearic acid/palmitic acid ratio and the oleic acid/palmitic acid ratio of a subject are calculated;
(2) a pharmaceutical composition containing as an active ingredient at least one selected from the group consisting of ω-3 polyunsaturated fatty acids as well as pharmaceutically acceptable salts and esters thereof is administered to the subject for a certain period of time;
(3) then, one or more selected from among the oleic acid/stearic acid ratio, the stearic acid/palmitic acid ratio and the oleic acid/palmitic acid ratio of the subject are calculated again;
(4) one or more selected from among the oleic acid/stearic acid ratio, the stearic acid/palmitic acid ratio and the oleic acid/palmitic acid ratio that were calculated before the administration of the pharmaceutical composition are compared with those calculated after the administration to make an evaluation of the subject's condition or therapeutic effects;
(5) the pharmaceutical composition is administered to the subject for a certain period of time based on the evaluation; and
(6) if desired or required, (3) through (5) as above are repeated;
(7) so as to prevent/ameliorate or treat non-alcoholic steatohepatitis.

Preferably, the pharmaceutical composition of the present invention is a pharmaceutical composition whose administration is repeated alternately with the evaluation of therapeutic effects, or of the severity of a disease, based on a plasma fatty acid composition ratio as an index.

In the case where the evaluation of therapeutic effects is to be made by comparing the fatty acid composition ratio as an index, the OA/SA ratio for instance, before treatment with that after treatment, prophylactic/ameliorative or therapeutic effects on NASH or NAFLD can be considered as obtained if the OA/SA ratio of a subject after a treatment for a certain period of time is reduced in value as compared with that before the treatment. Such evaluation also applies to the pharmaceutical composition of the present invention, that is to say, the treatment with the inventive composition can be considered as effective if the OA/SA ratio of a subject as determined after the administration of the composition for a certain period of time is reduced in value as compared with that before the administration. This holds true for the SA/PA and OA/PA ratios.

While the evaluation of therapeutic effects may be made using any one out of the OA/SA, SA/PA and OA/PA ratios as an index, it is desirable to use two or more of these fatty acid ratios and, in that case, include the OA/SA ratio in the ratios used.

Unless otherwise specified, the fatty acid composition ratio as used in the present invention may be a composition ratio of fatty acids in any of the plasma, serum and liver. It is also possible indeed to use a fatty acid composition ratio in a specified fraction, such as LDL or VLDL in the blood. It, however, is desirable to use a composition ratio of fatty acids in the plasma or the serum because of the simplicity of measurement. Each fatty acid to be employed for the calculation of a fatty acid composition ratio is not particularly limited in unit of amount, that is to say, its amount may be expressed in mole, mole percent, a unit of weight, percent by weight, or the like. The sole unit, and the sole method of calculating fatty acid composition ratios should be used if the evaluation is to be made by the comparison of a fatty acid composition ratio over time. It is particularly desirable to calculate a fatty acid composition ratio from fatty acid amounts expressed in mole percent of the total amount of fatty acids. The weight/volume concentration (e.g., μg/ml), the mole/volume concentration (e.g., mol/L) or the like may also be used for the calculation.

In this description, the term “plasma fatty acid” refers to a plasma total fatty acid unless otherwise specified. It is also possible to use a plasma free fatty acid for the inventive index for the evaluation of the subject's condition or therapeutic effects. The term “liver fatty acid” refers to a liver total fatty acid unless otherwise specified. A liver free fatty acid may optionally be used.

The fatty acid composition may be determined by any method practicable by a person of ordinary skill in the art of the present invention, while it is particularly preferable to determine the composition according to a usual manner.

The plasma total fatty acid composition may be determined by collecting blood from a subject to separate the plasma, hydrolyzing the lipids as extracted by Folch's method or the like to generate free fatty acids, and preparing fractions of all the free fatty acids by gas chromatography and so forth to obtain quantitative values and compositional values, from which a fatty acid composition ratio can be calculated. A fatty acid composition ratio may also be calculated from values obtained for individual fatty acids by the total lipid fatty acid fractionation (feasible at a clinical testing service provider such as SRL, Inc.) as a routine for clinical testing.

The plasma free fatty acid composition may be determined by developing a TLC plate with respect to the lipids as extracted by Folch's method or the like so as to collect only the free lipids as scraped off the plate, and subsequently conducting hydrolysis, methylation, then fractionation by gas chromatography and so forth to obtain quantitative values and compositional values, from which a fatty acid composition ratio can be calculated. The method is also applicable to the serum.

A composition ratio of fatty acids in the liver may be determined according to a usual manner. For instance, Folch's method or the like is followed to extract lipids with chloroform/methanol (2:1 vol/vol) from 5 to 10 mg of liver tissue, then conduct fractionation by chromatography. As a matter of course, the determination or calculation methods as above are in no way limitative, and any method practicable by a person of ordinary skill in the art of the present invention is usable.

In the present invention, a pharmaceutical composition containing ω-3 PUFAs refers to a pharmaceutical composition containing as an active ingredient at least one selected from the group consisting of ω-3 polyunsaturated fatty acids (ω-3 PUFAs) as well as pharmaceutically acceptable salts and esters thereof.

If the evaluation of therapeutic effects is to be made by comparing a fatty acid composition ratio before the administration of a pharmaceutical composition for a certain period of time with that after the administration, the period of time for which the pharmaceutical composition is administered is not particularly limited, while it desirably lasts seven days to one year, preferably 14 days to nine months, more preferably one to six months, and even more preferably one to three months, especially just one month.

In many of the subjects to whom the inventive pharmaceutical composition containing ω-3 PUFAs is administered, the OA/SA ratio of the subject tends to be reduced, whereupon the reduction rate of the OA/SA ratio is high for about one to two months after the start of administration, then the reduction rate decreases gradually. In this description, the reduction rate of a fatty acid composition ratio refers to the rate of the reduction in the relevant fatty acid composition ratio in a certain period of time. As an example, the reduction rate of the OA/SA ratio in one month is calculated by the equation: reduction rate (%)=[(OA/SA ratio before administration)−(OA/SA ratio after administration for one month)]/OA/SA ratio before administration×100.

In a preferred embodiment of the treatment method of the present invention, the inventive pharmaceutical composition containing ω-3 PUFAs is administered for one to two months, and the values of one or more out of the plasma OA/SA, SA/PA and OA/PA ratios of a subject before the administration of the composition are compared with those after the administration. If any of the OA/SA, SA/PA and OA/PA ratios is reduced after the administration as compared with that before the administration, it can be considered that therapeutic effects on NASH are gained in the subject from the pharmaceutical composition of the invention.

In another preferred embodiment of the treatment method of the present invention, the plasma OA/SA ratio of a subject is determined over time, one month and two months after the start of administration of the inventive pharmaceutical composition containing ω-3 PUFAs, for instance, and the values of the OA/SA ratio thus obtained are compared with those before the start of administration. In order that therapeutic effects on NASH are considered as gained, the reduction rate of the OA/SA ratio should desirably be 1% or higher, more desirably 2% or higher, and even more desirably 3% or higher, especially not lower than 5%, in one month after the start of administration. If the reduction rate is higher in one to two months after the start of administration, it is expected more readily that therapeutic effects on NASH are gained in the subject from the pharmaceutical composition of the invention, so that the administration of the inventive pharmaceutical composition containing ω-3 PUFAs is further continued. The reduction rate of the OA/SA ratio begins to decrease about two months after the start of administration. It is desirable to continue administering the inventive composition from then on so as to maintain a reduced value of the OA/SA.

In the subject to whom ω-3 PUFAs have been administered for at least several months, the plasma fatty acid composition, which initially varied after the start of administration, may show no variation more. For instance, the OA/SA ratio may be invariant for a certain period of time in the course of administration of ω-3 PUFAs. In that case, the evaluation of therapeutic effects should be made by comparing the measured values of fatty acids with those obtained before treatment, or conducting another test in combination.

In a preferred embodiment of the treatment method of the present invention, if one or more, or two or more, out of the OA/SA, SA/PA and OA/PA ratios after the administration of the pharmaceutical composition of the invention for one month have been increased or remain invariant as compared with those before the administration, it is desirable for enhanced therapeutic effects to carry out (1) increase in doses of ω-3 PUFAs, (2) addition or change of another drug, (3) addition or change of diet therapy, (4) addition or change of exercise therapy, and so forth.

In contrast, if one or more, or two or more, out of the OA/SA, SA/PA and OA/PA ratios of the subject have been reduced as compared with those before the administration, therapeutic effects are obtained, and treatment is continued effectively.

In a preferred embodiment of the treatment method of the present invention, it is desirable to carry out the above measures for enhanced therapeutic effects also on the subject in whom the reduction rate of the OA/SA ratio is low in two months after the start of administration of the inventive pharmaceutical composition. The phrase “the reduction rate of the OA/SA ratio is low in two months” means that the reduction rate is 0.5% or lower in two months, although not limited thereto.

In other words, a preferred embodiment of the present invention is a method of preventing/ameliorating or treating non-alcoholic steatohepatitis, in which the inventive pharmaceutical composition containing ω-3 PUFAs is administered to a subject using the reduction rate of the OA/SA ratio of a subject in one month after the start of administration of the pharmaceutical composition containing ω-3 PUFAs as an index for the evaluation of the subject's condition or therapeutic effects.

A more preferred embodiment is a method of preventing/ameliorating or treating non-alcoholic steatohepatitis, in which the inventive pharmaceutical composition containing ω-3 PUFAs is administered to a subject using the reduction rate of the OA/SA ratio of a subject in one month after the start of administration of the pharmaceutical composition containing ω-3 PUFAs as an index for the evaluation of the subject's condition or therapeutic effects so that the reduction rate of the OA/SA ratio in one month may be 1% or higher.

A particularly preferred embodiment is a method of preventing/ameliorating or treating non-alcoholic steatohepatitis, in which the inventive pharmaceutical composition containing ω-3 PUFAs is administered to a subject using the reduction rate of the OA/SA ratio in one month after the start of administration of the pharmaceutical composition containing ω-3 PUFAs as an index for the evaluation of the subject's condition or therapeutic effects so that the reduction rate of the OA/SA ratio in one month may be 1% or higher, and, after the reduction rate of the OA/SA ratio has decreased, the pharmaceutical composition containing ω-3 PUFAs is administered so that the reduction rate may be kept.

Another preferred embodiment of the present invention is a pharmaceutical composition containing ω-3 PUFAs for use in the above methods of preventing/ameliorating or treating non-alcoholic steatohepatitis.

According to the present invention, by observing the fatty acid change in a relatively short term such as one to three months, the subject's condition can be grasped, and it can be determined whether or not the treatment is effective at present, whether or not the treatment is adequate at present, whether or not the treatment needs to be changed, and whether or not the treatment is expected to become effective. The present invention makes it possible to provide a treatment more appropriate to a subject because it allows an earlier evaluation of therapeutic effects than other tests or marker changes reflecting the treatment of NASH.

The prevention/amelioration or treatment method of the present invention is desirably continued for at least one month, preferably three months or longer, more preferably six months or longer, and even more preferably one year or longer, especially at least three years. It is most preferable to continue the method over a period of not less than five years. Preferably, the prevention/amelioration or treatment method of the present invention is continued until it is determined using as an index other test or marker as described later that the treatment can be terminated.

The pharmaceutical composition of the present invention is desirably administered for at least one month, preferably three months or longer, more preferably six months or longer, and even more preferably one year or longer, especially at least three years. It is most preferable to administer the composition over a period of not less than five years. Preferably, the pharmaceutical composition of the present invention is administered until it is determined using as an index other test or marker as described later that the treatment can be terminated.

If the pharmaceutical composition of the invention is to be administered to a subject based on the evaluation of therapeutic effects, (1) modification in doses of ω-3 PUFAs, (2) addition of another drug, modification in dose of another drug, stop or withdrawal of another drug, (3) addition or change of diet therapy or exercise therapy, and so forth may be carried out as desired or required in order to attain the subject's condition or therapeutic effects which are desired for the treatment of NASH.

The subject's condition or therapeutic effects are determined by the subject him-/herself or a doctor from physical and mental conditions of the subject in a non-limitative manner. If the NAS scoring of Kleiner et al. is employed, for instance, determinations are made in accordance with the improvement in scores. Kleiner et al. gave scores to three histological findings about NAFLD liver, namely, the degrees of fatty liver (steatosis), hepatocellular ballooning, and parenchymatous inflammation (lobular inflammation) (NAFLD activity score: NAS), and defined NAS of 5 or more as indicating NASH. The subject's condition or therapeutic effects which are desired are preferably represented by a decrease of NAS by 1 or more, more preferably by a decrease by 2, with a decrease of NAS to 4 or less being even more preferable.

Matteoni et al. classified NAFLD on the basis of pathological findings into four types, namely, type 1: simple fatty liver, type 2: fatty liver plus lobular inflammation, type 3: fatty liver plus hepatocellular ballooning (ballooning degeneration of hepatocytes), and type 4: fatty liver, hepatocellular ballooning, plus liver fibrosis or Mallory bodies, and proposed that types 3 and 4 diseases be diagnosed as NASH. Brunt et al. proposed that pathological findings about NASH be evaluated and classified by inflammation grading and fibrosis staging, and their method is widely used for pathological classification of NASH. The subject's condition or therapeutic effects may be determined by using either of such classifications. In that case, the subject's condition or therapeutic effects which are desired are represented by a remission of a type 3 disease to a type 2 one, or of a type 4 disease to a type 3 one, according to the classification of Matteoni et al., or an improvement in grade of inflammation and/or stage of fibrosis according to the classification of Brunt et al.

In its second aspect, the present invention provides a method of preventing/ameliorating or treating non-alcoholic steatohepatitis or NAFLD, which includes starting administration of the pharmaceutical composition of the present invention to a subject having a plasma oleic acid/stearic acid ratio of not less than 3, continuing the administration until the plasma oleic acid/stearic acid ratio is reduced to below 2.6, and further continuing the administration so that the plasma oleic acid/stearic acid ratio is maintained below 2.6. In the method, the plasma fatty acid composition ratio is to be calculated based on the plasma total fatty acid composition, whereupon the unit of measurement to be used is the mole or mole percent. Calculation from the values obtained by a twenty-four plasma fatty acid fractionation test is preferable.

In its third aspect, the present invention provides a method of preventing/ameliorating or treating non-alcoholic steatohepatitis or NAFLD, which includes using a test or a marker, or a combination thereof as an index for the evaluation of the subject's condition or therapeutic effects to administer a pharmaceutical composition containing ω-3 PUFAs to the subject. In the method, either the test or marker to be used is not particularly limited, with a possible marker being a fatty acid composition ratio, or a fatty acid level in mole percent. For instance, a pharmaceutical composition containing ω-3 PUFAs may be administered to a subject having a plasma oleic acid level in mole percent higher than the average for healthy people, using a measured value of oleic acid in plasma as an index.

Examples of the test or marker to be used include diagnostic imaging (e.g., ultrasonography (echography), CT, MRI), insulin resistance test (e.g., HOMA-IR), as well as blood insulin level, after-meal glucose level, BMI, oxidative stress marker in blood (e.g., thioredoxin, malondialdehyde, 4-hydroxynonenal, nitric oxide), 8-isoprostane, adipocytokine (e.g., adiponectin, TNFα, leptin, MCP1, resistin), fibrosis marker (e.g., hyaluronic acid, type IV collagen, procollagen III polypeptide (PIIIP), TIMP-1 (tissue inhibitor of metalloproteinase-1), CTGF (connective tissue growth factor)), sTNF-R, platelet count, serum ferritin, serum iron, ALT, AST, ALT/AST ratio, γ-GTP, ALP (alkaline phosphatase), KICG, high sensitivity CRP, triglyceride (TG), LDL-C, HDL-C, total cholesterol, free fatty acid (FFA), phospholipid, albumin, total protein, total bilirubin, kininogen, 17-OH-oleic acid [omega-1-OH-oleic acid], 18-OH-oleic acid [omega-oleic acid], PDGF-A, PDGF-B, IL-1β, IL-2, and IL-6. The testings can be carried out by any method practicable by a person of ordinary skill in the art of the present invention. Preferably, they can be carried out according to a usual manner.

Particularly preferable as an index for the evaluation of the therapeutic effects of the inventive pharmaceutical composition are AST, ALT, ALP, total bilirubin, thioredoxin, ferritin, MCP1, hyaluronic acid, type IV collagen, TIMP-1, CTGF, TG, albumin, total protein, 17-OH-oleic acid [omega-1-OH-oleic acid], 18-OH-oleic acid [omega-oleic acid], and so forth. Combination with diagnostic imaging is more preferable.

It is desirable that the tests and markers as above are selected in combination as appropriate to the subject's condition.

For instance, in a subject having a disease classified as type 4 (fatty liver, hepatocellular ballooning, plus liver fibrosis or Mallory body-forming liver fibrosis) according to the classification of Matteoni et al., the evaluation of therapeutic effects is to be made using one or more out of hyaluroninc acid, type IV collagen, TIMP-1, albumin, total protein, ALP and total bilirubin in combination with another testing (e.g., HOMA-IR, blood insulin level, BMI, TG).

In a subject having a disease classified as type 3 (fatty liver plus hepatocellular ballooning) according to the classification of Matteoni et al., the evaluation is preferably made using MCP1, AST, ALT, thioredoxin, ferritin, platelet count, blood insulin level, BMI, or the like.

Test values obtained from the above preferable tests and markers are gradually improved reflecting the therapeutic effectiveness of ω-3 PUFAs against NASH, namely, improvement in pathological findings and so forth, so that the measurements performed in the tests or on the markers make the need of liver biopsy reduced, leading to a reduction in burden on subjects.

The tests and markers as above are improved in test value gradually as the therapeutic effects on NASH are gained from ω-3 PUFAs, and are effective as such at grasping the progress of NASH treatment. In addition, the tests and markers are usable to set the index to (target value for) the cure of a disease which allows the termination of treatment. The target value which permits considering a disease as cured can be set at any of normal values for individual testings.

The pharmaceutical composition of the present invention is administered using as an index such a test or marker as mentioned above in order to attain a targeted marker value. The pharmaceutical composition of the present invention is the pharmaceutical composition containing ω-3 PUFAs as active ingredients that is adapted to improve findings from imaging, test values or marker values obtained in a patient with NASH or NAFLD. The treatment method of the present invention is the method of preventing/ameliorating or treating NASH in which a pharmaceutical composition containing ω-3 PUFAs is administered using as an index such a test or marker as mentioned above in order to attain the target value.

In its fourth aspect, the present invention provides a pharmaceutical composition containing ω-3 PUFAs as active ingredients which is adapted to improve a plasma, serum or liver fatty aced composition ratio of a patient with NASH or NAFLD. Also provided is a pharmaceutical composition containing ω-3 PUFAs as active ingredients which is adapted to improve a plasma free fatty acid composition ratio of a patient with NASH or NAFLD.

A fatty acid composition ratio, the OA/SA ratio for instance, of a subject is considered as improved if the value of the OA/SA ratio after the administration of a pharmaceutical composition for a certain period of time is reduced as compared with that before the administration. This holds true for the SA/PA and OA/PA ratios. The pharmaceutical composition according to the fourth aspect of the present invention is prepared and used in a similar manner to the pharmaceutical composition as used in the treatment method according to the first aspect.

In its fifth aspect, the present invention provides a method of selecting the patient with NASH or NAFLD who is expected to be responsive to the treatment with a pharmaceutical composition containing ω-3 PUFAs as active ingredients. The patient with NASH (or NAFLD) who is expected to be responsive to ω-3 PUFAs is the subject whose OA/SA ratio as a plasma fatty acid composition ratio is high, irrespective of the presence or absence of physical symptom. The subject has a high OA/SA ratio as compared with the mean OA/SA ratio for healthy people, that is to say, is a human with a plasma OA/SA ratio of not less than 1.5, preferably 2 or more, especially 3 or more. In this regard, the unit of measurement to be used is the mole or mole percent.

The patient with NASH or NAFLD who is expected to be responsive to the treatment with the inventive pharmaceutical composition containing ω-3 PUFAs as active ingredients is the subject in whom, when blood fatty acid levels are measured before the start of administration of the pharmaceutical composition containing ω-3 PUFAs as active ingredients and one to three months after the start of the administration so as to make a comparison of one or more selected from among the OA/SA, SA/PA and OA/PA ratios, the blood fatty acid ratios are reduced.

Desirably, the above blood fatty acid ratios of the subject have high reduction rates, preferably the desirable reduction rates as mentioned before. In such a subject, therapeutic effects gained from the pharmaceutical composition of the present invention will be significant.

It is preferable to continue administering the pharmaceutical composition of the present invention to the subject as described above.

In other words, preferred is the method of preventing/ameliorating or treating non-alcoholic steatohepatitis, in which,

(1) one or more selected from among the plasma oleic acid/stearic acid ratio, the plasma stearic acid/palmitic acid ratio and the plasma oleic acid/palmitic acid ratio of a subject are calculated,
(2) a pharmaceutical composition containing as an active ingredient at least one selected from the group consisting of ω-3 polyunsaturated fatty acids as well as pharmaceutically acceptable salts and esters thereof is administered to the subject for a certain period of time,
(3) then, one or more selected from among the plasma oleic acid/stearic acid ratio, the plasma stearic acid/palmitic acid ratio and the plasma oleic acid/palmitic acid ratio of the subject are calculated again,
(4) one or more out of the plasma oleic acid/stearic acid ratio, the plasma stearic acid/palmitic acid ratio and the plasma oleic acid/palmitic acid ratio as obtained before the administration of the pharmaceutical composition are compared with those obtained after the administration and, if the latter are reduced in value, the administration of the pharmaceutical composition to the subject is continued.

Hepatic lesion (NASH or NAFLD) may develop in the subject in whom one or more out of the plasma OA/SA, SA/PA and OA/PA ratios are periodically determined, and are increased in value as compared with those determined previously.

An early start can be made on treating or prophylactically treating such a subject, irrespective of whether or not the determined values are beyond normal ranges. Such a subject is expected to be responsive to ω-3 PUFAs.

In other words, the patient with NASH or NAFLD who is expected to be responsive to the treatment with the inventive pharmaceutical composition containing ω-3 PUFAs as active ingredients is the subject in whom one or more out of the plasma OA/SA, SA/PA and OA/PA ratios are increased in value as compared with those determined within the preceding one year. Particularly to be selected is the subject whose OA/SA ratio as a plasma fatty acid composition ratio is increased by not less than 0.5, preferably 1.0 or more, more preferably 1.5 or more, as compared with that determined previously. In this regard, a previous determination is desirably carried out within the preceding one year, more desirably within the preceding six months. It is preferable that the subject to be selected is specified not only from the indices as described above but the variation in test values on liver functions. To the subject thus selected, any drug having therapeutic effects on NASH is administered. While the drug to be administered may be selected as appropriate to the physical condition of the subject, it is preferable to use ω-3 PUFAs. Upon the administration of a drug, the dose may be modified, and another drug may be added, under observation of the physical condition of a patient.

The patient with NASH or NAFLD who is expected to be responsive to the treatment with the inventive pharmaceutical composition containing ω-3 PUFAs as active ingredients is a subject having high blood free fatty acid levels. Blood free fatty acid levels can generally be measured by any method practicable by a person of ordinary skill in the art of the present invention. The measurement may be entrusted to a clinical testing service provider such as SRL, Inc. The reference values are each defined to be 140 to 850 (μEq/L). The blood free fatty acid levels are liable to be affected by diet, so that it is desirable to measure them under fixed conditions brought about by an overnight fasting, for instance. Preferably, the subject has been kept having high blood free fatty acid levels over a long period of time, six months or longer for instance, and more preferably one year or longer. A high blood free fatty acid level refers to a blood free fatty acid level higher than the mean blood free fatty acid level for healthy people.

The patient with NASH or NAFLD who is expected to be responsive to the treatment with the inventive pharmaceutical composition containing ω-3 PUFAs as active ingredients is the subject whose blood triglyceride (TG) level is reduced by administering the pharmaceutical composition of the present invention. Particularly preferred is the subject whose blood TG level is above the reference value before the start of administration of the inventive pharmaceutical composition, and is essentially not reduced for about one to six months after the start of administration, but then reduced gradually.

The patient with NASH or NAFLD who is expected to be responsive to the treatment with the inventive pharmaceutical composition containing ω-3 PUFAs as active ingredients is a subject suffering from a subject with early stage of NASH. Exemplary subjects include those with a stage 1 or 2 disease according to the fibrosis staging of Brunt et al. Brunt et al. evaluated and classified pathological findings about NASH by inflammation grading and fibrosis staging. In particular, the fibrosis staging has been done as follows.

Stage 1: pericentral fibrosis (pericellular), local or comprehensive.

Stage 2: pericentral fibrosis and periportal fibrosis.

Stage 3: pericentral fibrosis and periportal fibrosis with bridge formation.

Stage 4: cirrhosis.

The patient with NASH or NAFLD who is expected to be responsive to the treatment with the inventive pharmaceutical composition containing ω-3 PUFAs as active ingredients is a subject with a high degree of oxidation in the body. Exemplary subjects include those with an accelerated production of reactive oxygen species (ROS), and those with iron overload. The increase in serum ferritin, the serum thioredoxin level, and the increase in 8-isoprostane level can be used to specify such subjects.

The patient with NASH or NAFLD who is expected to be responsive to the treatment with the inventive pharmaceutical composition containing ω-3 PUFAs as active ingredients is a subject suffering from a severe inflammation. Such a subject can be specified by measuring MCP1, high sensitivity CRP, and so forth.

The patient with NASH or NAFLD who is expected to be responsive to the treatment with the inventive pharmaceutical composition containing ω-3 PUFAs as active ingredients is a subject with an increase in AST or ALT.

The patient with NASH or NAFLD who is expected to be responsive to the treatment with the inventive pharmaceutical composition containing ω-3 PUFAs as active ingredients may also be a subject having a single nucleotide polymorphism (SNP) in the adiponectin gene or TNF gene.

The treatment method of the present invention is also considered as the method of treating NASH, in which the subject expected to be responsive to ω-3 PUFAs as described above is selected, and a pharmaceutical composition containing ω-3 PUFAs such as EPA-E as active ingredients is administered using as an index a fatty acid composition ratio of the subject, another test or marker, or a combination thereof, and in combination with another drug for combined application, dietary restriction, and so forth as required for treatment, until the target test value is attained.

In its sixth aspect, the present invention provides a pharmaceutical composition for amelioration or treatment of NASH containing ω-3 PUFAs as active ingredients, which is suitably used for the subject diagnosed by liver biopsy as having NASH whose plasma triglyceride (TG) is higher in value than normal (hypertriglyceridemia). According to the criteria defined in the Guidelines for Prevention of Atherosclerotic Cardiovascular Diseases, 2007 edition (edited and published by Japan Atherosclerosis Society), a high plasma TG, or hypertriglyceridemia, refers to the fact that the triglyceride level as obtained from the blood collected at fasting is not less than 150 mg/dL.

The pharmaceutical composition as above is also suitable for “a subject suffering from dyslipidemia.” According to the criteria defined in the Guidelines for Prevention of Atherosclerotic Cardiovascular Diseases, 2007 edition (edited and published by Japan Atherosclerosis Society), the term “dyslipidemia” means a condition applying to at least one out of high LDL cholesterolemia, namely the condition in which the serum LDL cholesterol level as obtained from the blood collected at fasting is not less than 140 mg/dL, low HDL cholesterolemia, namely the condition in which the serum HDL cholesterol level as obtained from the blood collected at fasting is less than 40 mg/dL, and hypertriglyceridemia, namely the condition in which the serum triglyceride level as obtained from the blood collected at fasting is not less than 150 mg/dL.

According to the ATP III Guidelines At-A-Glance Quick Desk Reference published by the National Institutes of Health (NIH) of the United States in May of 2001, the term “dyslipidemia” may mean a condition applying to at least one out of the condition in which the serum LDL cholesterol level is not less than 130 mg/dL, the condition in which the serum total cholesterol level is not less than 200 mg/dL, and the condition in which the serum HDL cholesterol level is less than 40 mg/dL.

In its seventh aspect, the present invention provides a pharmaceutical composition for amelioration or treatment of NASH containing ω-3 PUFAs as active ingredients, which is suitably used for “a subject diagnosed by liver biopsy as having NASH” or “the subject determined by diagnostic imaging or the like as having fatty liver in whom the increase in AST or ALT is observed.”

During the determination of “fatty liver,” the presence of fat droplets in one-third or more of hepatocytes is diagnosed as fatty liver, whereupon the presence of fat droplets in not less than 10% hepatocytes is diagnosed as fatty liver in a broad sense. The determination of fatty liver is generally carried out by diagnostic imaging, such as ultrasonography, CT and MRI, although not limited thereto. For instance, the finding called “bright liver” as obtained from the echo intensity may be diagnosed as fatty liver.

The increase in AST (also referred to as GOT) or ALT (also referred to as GPT) is generally considered as an increase to a value two to four times as large as the upper limit of the normal value range. While the normal value for AST may optionally be specified to about 10 to 40 IU/L, and that for ALT to about 5 to 40 IU/L, normal values (reference values) are merely measures for diagnosis, which may vary with medical facilities. Diagnosis of NASH may be made by liver biopsy even if both AST and ALT are normal in value.

The pharmaceutical composition of the present invention suitably used for such a subject as above is not particularly limited in the time to start administration, while it is preferable to start administering the composition within three years, more preferably one year, after the diagnosis was made for the first time.

In its eighth aspect, the present invention provides a pharmaceutical composition for amelioration or treatment of NASH containing ω-3 PUFAs as active ingredients, which reduces AST or ALT in the subject diagnosed by liver biopsy as having NASH in whom the increase in AST or ALT is observed. Reduction in AST or ALT may be achieved by making the value of AST or ALT smaller than the test value before the start of administration of the composition, and the value of AST or ALT is preferably reduced to two-thirds, more preferably a half, of the test value before the start of the administration, with the even more preferred being a reduction to a magnitude considered as normal (magnitude of the reference value).

The pharmaceutical composition of the present invention is used as described above.

In its ninth aspect, the present invention provides a method of using the OA/SA ratio, SA/PA ratio, OA/PA ratio in the plasma, serum or liver of a subject, another test or marker, or a combination thereof as a marker for NASH occurrence instead of the affirmative diagnosis of NASH by liver biopsy. In the method, the fatty acid composition ratios and another tests or markers can be used in combination as described with respect to the first to third aspects of the present invention to increase the reliability of diagnosis.

In its tenth aspect, the present invention provides a method of finding a subject having the fatty liver which is highly liable to transit to NASH by using a fatty acid composition ratio of the subject, another test or marker, or a combination thereof as an index. If the OA/SA, SA/PA or OA/PA ratio of the subject is continuously high in value, for instance, the risk of NASH onset is great. The risk of NASH onset is increased as a high value of the OA/SA ratio lasts one month or longer, three months or longer, six months or longer, or even one year or longer.

In its eleventh aspect, the present invention provides a method of preventing NASH, in which a subject having the fatty liver which is highly liable to transit to NASH is found, and the time to start treatment is chosen using test values as an index so as to start treatment. The start of prevention of NASH is desirably decided for the subject in whom a high value of the OA/SA ratio lasts one month or longer, three months or longer, six months or longer, or even one year or longer, or the OA/SA ratio is being increased in value as compared with that determined previously, whereupon another test or marker is also taken into account.

In its twelfth aspect, the present invention provides a pharmaceutical composition containing ω-3 PUFAs as active ingredients, which ameliorates liver fibrosis due to NASH. The index for amelioration may be the OA/SA ratio. The pharmaceutical composition of the present invention is administered preferably in combination with a diet therapy, using the plasma OA/SA ratio as an index so that the value of the ratio may be reduced from that determined previously.

In other words, the inventive composition as above is a pharmaceutical composition containing ω-3 PUFAs as active ingredients, which ameliorates liver fibrosis due to NASH, and which is administered preferably in combination with a diet therapy so that the value of the plasma OA/SA ratio may be reduced as compared with that determined previously.

The subject as a target is preferably a subject already suffering from liver fibrosis. Preferred examples include a subject having a disease classified as from type 3 late stage, to type 4 (fatty liver plus hepatocellular ballooning, plus liver fibrosis or Mallory bodies) according to the classification of Matteoni et al. The subject in whom occurrence of liver fibrosis is determined from, for instance, a fibrosis marker (e.g., hyaluronic acid, type IV collagen, TIMP-1) is preferable.

In its thirteenth aspect, the present invention provides a pharmaceutical composition containing ω-3 PUFAs as active ingredients, which is adapted to suppress liver fibrosis in a subject having fatty liver. The pharmaceutical composition containing ω-3 PUFAs as active ingredients is also adapted to suppress transition to liver cancer in a patient with NAFLD or NASH. The composition can be used in the inventive method of preventing/ameliorating or treating non-alcoholic steatohepatitis.

In its fourteenth aspect, the present invention provides a method of advertising the NASH or NAFLD prevention/amelioration or treatment method or subject management method of the present invention that uses ω-3 PUFAs. The method is also adapted to advertise the pharmaceutical composition containing ω-3 PUFAs which is used in the NASH or NAFLD treatment method or subject management method of the present invention.

In the method, information on the NASH or NAFLD treatment method or subject management method of the present invention is provided to a doctor or a subject. To be more specific: It can be informed for advertisement that the therapeutic effects of ω-3 PUFAs on NASH can be predicted or evaluated on the basis of the variation in a specified fatty acid composition ratio of a subject in a certain period of time, and so forth. The means for advertisement is in no way limited, and examples include distribution of pamphlets or electronic media, and information provision through the Internet.

The best configurations for the implementation of the present invention are as detailed above. As a matter of course, any favorable modification may be made without departing from the scope of the invention.

Next, the terms as used in the above description are explained in more detail.

Polyunsaturated fatty acids (PUFAs) are defined as those fatty acids each of which has a plurality of carbon-carbon double bonds in the molecule, and classified as ω-3 fatty acids, ω-6 fatty acids, and so forth in accordance with the positions of double bonds. Exemplary ω-3 PUFAs include α-linolenic acid, EPA, and docosahexaenoic acid (hereafter referred to as DHA). Unless otherwise specified, the term “PUFAs” as used herein implies not only polyunsaturated fatty acids but pharmaceutically acceptable salts as well as derivatives such as esters, amides, phospholipids and glycerides of polyunsaturated fatty acids.

The ω-3 PUFAs to be used in the present invention may be synthetic, semisynthetic or natural products, or may be in the form of natural oil containing them. The term “natural product” as used herein means a product obtained from a natural oil containing ω-3 PUFAs by a conventional extraction or crude purification, or a product obtained by highly purifying such a product. The term “semisynthetic product” implies a polyunsaturated fatty acid produced by a microorganism or the like, and also implies the polyunsaturated fatty acid as such or the polyunsaturated fatty acid as a natural product which has been subjected to a chemical treatment such as esterification or transesterification. In the present invention, a single ω-3 PUFA or a combination of two or more ω-3 PUFAs may be used.

The ω-3 PUFAs to be used in the present invention are specifically exemplified by EPA, DHA, α-linolenic acid, as well as pharmaceutically acceptable salts and esters thereof. Examples of pharmaceutically acceptable salts and esters include salts with inorganic bases such as sodium salt and potassium salt, salts with organic bases such as benzylamine salt and diethylamine salt, salts with basic amino acids such as arginine salt and lysine salt, as well as alkyl esters such as ethyl ester and esters of mono-, di- and triglycerides. Preferred is ethyl ester, especially EPA-E and/or DHA-E. EPA-E (ethyl icosapentate ester) is particularly desirable.

The ω-3 PUFAs are not particularly limited in purity, while it is generally preferable that the ω-3 PUFAs comprise not less than 25% by weight, more preferably not less than 50% by weight, and even more preferably not less than 70% by weight, especially not less than 85% by weight, of the fatty acids contained in the inventive composition. In a particularly desirable embodiment, the inventive composition contains essentially no other fatty acids than ω-3 PUFAs.

In an exemplary case where EPA-E and DHA-E are to be used, the composition ratio EPA-E/DHA-E or the ratio of the (EPA-E+DHA-E) content to the total content of the fatty acids in the composition is not particularly limited, while the composition ratio EPA-E/DHA-E is preferably 0.8 or more, more preferably 1.0 or more, and even more preferably 1.2 or more. The combination of EPA-E and DHA-E is preferably of high purity, that is to say, as an example, the ratio of the (EPA-E+DHA-E) content to the total content of the fatty acids and derivatives thereof in the composition is preferably not less than 40% by weight, more preferably not less than 55% by weight, even more preferably not less than 84% by weight, especially not less than 96.5% by weight. In this connection, it is desirable that any long-chain saturated fatty acid content is low, and any ω-6 fatty acid, particularly arachidonic acid, is low in content even though it is a long-chain unsaturated fatty acid, whereupon a content lower than 2% by weight, in particular lower than 1% by weight, is preferred.

The EPA-E and/or DHA-E as used in the composition of the present invention is accompanied by less impurities unfavorable to cardiovascular events, such as saturated fatty acids and arachidonic acid, as compared with fish oils or concentrates thereof, and can exert effective actions without overnutrition or excess intake of vitamin A. In addition, the EPA-E and/or DHA-E, as being an ester, has a high oxidation stability as compared with the fish oils which are chiefly in the form of triglyceride, and allows a composition to be made adequately stable by adding a conventional antioxidant.

The EPA-E to be used may be in the form of high purity EPA-E (at least 96.5% by weight pure)-containing soft capsules available in Japan as a therapeutic agent against arteriosclerosis obliterans (ASO) and hyperlipidemia (trade name, EPADEL; manufactured by MOCHIDA PHARMACEUTICAL CO., LTD.). The mixture of EPA-E and DHA-E may be LOVAZA (manufactured by GlaxoSmithKline plc; soft capsules containing ca. 46.5% by weight EPA-E and ca. 37.5% by weight DHA-E) commercially available in the USA as a therapeutic agent against hypertriglyceridemia.

Purified fish oils may also be used as ω-3 PUFAs. Monoglycerides, diglycerides, triglycerides of ω-3 PUFAs, and combinations thereof are also included in preferable examples. A variety of commercially available products containing ω-3 PUFAs as well as salts and esters thereof, such as Incromega F2250, F2628, E2251, F2573, TG2162, TG2779, TG2928, TG3525 and E5015 (Croda International PLC, Yorkshire, England), and EPAX6000FA, EPAX5000TG, EPAX4510TG, EPAX2050TG, EPAX7010EE, K85TG, K85EE and K80EE (Pronova Biopharma, Lysaker, Norway), are also usable.

In the pharmaceutical composition and treatment method of the present invention, ω-3 PUFAs may be used or applied in combination with another drug. Another drug to be used in the present invention is not particularly limited, while preferable examples include those which do not weaken the effects of the present invention, such as a liver protection drug, a hypoglycemic agent, an antihyperlipidemic agent, an antihypertensive agent, an antioxidant, and an antiinflammatory agent.

The liver protection drug is exemplified by ursodeoxycholic acid and betaines. Examples of the hypoglycemic agent include biguanides such as metformin, insulin and insulin derivatives, sulfonylurea drugs such as tolbutamide, gliclazide, glibenclamide and glimepiride, fast-acting insulin secretion stimulators such as nateglinide, repaglinide and mitiglinide, α-glucosidase inhibitors such as acarbose, voglibose and miglitol, as well as thiazolidines such as pioglitazone, rosiglitazone and troglitazone. Examples of the antihyperlipidemic agent include HMG-CoA reductase inhibitors such as pravastatin, simvastatin, atorvastatin, fluvastatin, pitavastatin, rosuvastatin and cerivastatin, fibrate drugs such as simfibrate, clofibrate, clinofibrate, bezafibrate and fenofibrate, lipase inhibitors such as orlistat, as well as ezetimibe. Examples of the antihypertensive agent include angiotensin-converting enzyme inhibitors such as captopril, alacepril, imidapril, enalapril, cilazapril, temocapril, delapril, lisinopril and benazepril, angiotensin receptor blockers such as losartan, valsartan, candesartan, telmisartan, olmesartan, irbesartan and eprosartan, renin inhibitors such as aliskiren, as well as calcium antagonists such as amlodipine, nifedipine, benidipine, nicardipine, nilvadipine, cilnidipine, azelnidipine, manidipine, nitrendipine, barnidipine, nisoldipine, efonidipine, felodipine, aranidipine, diltiazem, verapamil and bepridil. Examples of the antioxidant include vitamins such as vitamin C and vitamin E, N-acetylcysteine, and probucol. Examples of the antiinflammatory agent include cytokine production suppressors such as pentoxifylline, leukotriene receptor antagonists, leukotriene biosynthesis inhibitors, NSAIDs, COX-2 specific inhibitors, M2/M3 antagonists, steroids such as corticosteroid and prednisolone farnesylate, Hi (histamine) receptor antagonists, as well as aminosalicylates such as salazosulfapyridine and mesalazine. Exemplary immunosuppressants include azathioprine, 6-mercaptopurine, and tacrolimus. Exemplary antiviral agents against hepatitis C virus (HCV) include interferons, protease inhibitors, helicase inhibitors, and polymerase inhibitors.

In the present invention, prevention should be construed not only as preventing the onset of a disease but delaying the onset and reducing the incidence rate. In the present invention, amelioration should be construed as improving not only some parameter or other of a disease but the subjective symptoms or quality of life of a patient. In the present invention, treatment should be construed not only as administering a drug to the patient who has already developed a disease but administering a drug to a patient with a high risk of developing a disease as a prophylactic treatment.

In the present invention, “application of active ingredients in combination” or “combined application of active ingredients” includes application of a combination of active ingredients, that is to say, administering ω-3 PUFAs and another drug as a formulation containing both, and administering ω-3 PUFAs and another drug as separate formulations simultaneously, or separately with a certain time lag, are included therein. In the mode of administration in which ω-3 PUFAs and another drug are administered “as separate formulations simultaneously, or separately with a certain time lag,” included are (1) administration of a composition containing another drug as an active ingredient to the subject to whom ω-3 PUFAs are to be administered, and (2) administration of a composition containing ω-3 PUFAs as active ingredients to the subject to whom another drug is to be administered. Moreover, although the drugs “applied in combination” are not necessarily limited to concurrently existing in the body of a subject, in the blood for instance, “application of active ingredients, or drugs, in combination” is defined in the present invention as the application method in which one drug is administered while the actions or effects of the other drug are exerted in the body of a subject. Such an application method makes it possible to prevent/ameliorate or treat a NAFLD- or NASH-associated disease effectively by using the composition of the present invention. With respect to this application method, it is preferable that the drugs are concurrently present in the body of a subject, in the blood for instance, and that one drug is administered to a subject within 24 hours after the administration of the other.

In terms of the active ingredients of the inventive pharmaceutical composition, the mode of application in combination is not particularly limited as long as the active ingredients are combined with each other. The following are exemplary modes: (1) The active ingredients are formulated at a time, and the single formulation thus obtained is administered. (2) The active ingredients are formulated separately, and the two formulations thus obtained are combined together into a kit or kept separate, and administered simultaneously from one and the same dosage route. (3) The active ingredients are formulated separately, and the two formulations thus obtained are combined together into a kit or kept separate, and administered separately with a certain time lag from one and the same dosage route. (4) The active ingredients are formulated separately, and the two formulations thus obtained are combined together into a kit or kept separate, and administered simultaneously from different dosage routes (from different sites of one and the same subject). (5The active ingredients are formulated separately, and the two formulations thus obtained are combined together into a kit or kept separate, and administered separately with a certain time lag from different dosage routes (from different sites of one and the same subject).

In the case of separate administration with a certain time lag, ω-3 PUFAs may be administered prior to another drug, or vice versa, for instance. In the case of simultaneous administration, the drugs may or may not be mixed together immediately before administration if the dosage route is one and the same. It is also possible to administer the drugs at different periods by design for various purposes. To be more specific: One drug may be administered and allowed to act when the effects of the other drug, which has previously been administered, begin to be exerted or are being fully exerted. Alternatively, one drug may be made into an extended release form to administer it once a day, whereupon the other drug may be administered more than one time, two or three times for instance, daily, or also once a day. It is preferable that both drugs are administered once a day and, moreover, the drugs are administered simultaneously or as combined together into a single formulation because the burden of medication on a subject is relieved, and an improved medication compliance and increased prophylactic/ameliorative or therapeutic effects as well as reduced side effects are expected. The drugs may be administered at a time so as to stop administration of one drug when the effects of both drugs begin to be exerted or are being fully exerted. If the administration of a drug is to be stopped, the drug may gradually be reduced in dose. Administering one drug during the withdrawal of the other is also available.

It is desirable that the therapeutic effects of the ω-3 PUFAs and another drug as applied in combination exceed the total effects of the ω-3 PUFAs and another drug as applied separately at the same doses as those upon the application in combination. In this regard, the therapeutic effects are not particularly limited as long as they are effects of preventing/ameliorating or treating a NAFLD- or NASH-associated disease or suppressing progression thereof to cirrhosis or liver cancer. Examples include, besides the improvement in fatty acid ratio as described before, the degree of liver fibrosis determined by an imaging test (e.g., echography, CT, MRI), liver biopsy, or from a fibrosis marker in the plasma (e.g., type IV collagen, hyaluronic acid, TIMP-1), the reduction in serum AST or ALT level, the reduction in AST/ALT ratio, the increase in adiponectin, the reduction in TNFα, the reduction in high sensitivity CRP or reduction in oxidative stress marker in blood (ferritin, thioredoxin), and the improvement in HOMA-IR. Another biochemical or pathological parameter or pathologic condition parameter related to NAFLD or NASH may also be used to monitor prophylactic/ameliorative or therapeutic effects.

The dosage and dosage periods of the ω-3 PUFAs and another drug used in the composition of the present invention are made effective amount of dosage and period to the expected actions of the drugs, and each modified as appropriate to the dosage form, dosage route, and frequency of administration per day of the relevant drug, the degree of a symptom, the body weight and age of a patient, and so forth.

In the case of oral administration, 0.1 to 10 g/day, preferably 0.3 to 6 g/day, more preferably 0.6 to 4 g/day, and even more preferably 0.9 to 2.7 g/day of EPA-E and/or DHA-E, for instance, is administered at a time or in two or three portions. Whether the entire amount is administered at a time or in portions may be determined as required. The dosage may be reduced in response to the dosage of another drug. Administration is preferably performed during meals or after meals, with an administration just after meals (within 30 minutes after a meal) being more preferred. The period of oral administration at the above dose will be at least one year, preferably two years or longer, more preferably 3.5 years or longer, and even more preferably 5 years or longer. It is desirable that administration be continued while a pathologic condition, biochemical index, or the like related to NASH remains, or the patient is under the situation where the risk of NASH onset and/or recurrence is great. Administration may also be performed every other day or two or three days a week, for instance, or with an optional drug withdrawal period for one day to about three months, preferably about one week to one month.

Another drug for the composition of the present invention is preferably used following the dosage regime for the relevant drug alone, while its dose may be modified as appropriate to the type, dosage form, dosage route, and frequency of administration per day of the drug, the degree of a symptom, the body weight, sexuality and age of a patient, and so forth. The dose may be reduced in response to the dose of ω-3 PUFAs. It is more preferable from the viewpoint of side effect relief that the daily dose is reduced as much as possible, and extended release tablets are utilized to achieve once-a-day administration. If another drug is orally administered with its dose as such, the dosage period will be at least one year, preferably two years or longer, more preferably 3.5 years or longer, and even more preferably 5 years or longer. It is desirable that administration be continued while a pathologic condition, biochemical index, or the like related to NASH remains, or the patient is under the situation where the risk of NASH onset and/or recurrence is great. Administration may also be performed every other day or two or three days a week, for instance, or with an optional drug withdrawal period for one day to about three months, preferably about one week to one month.

During the application of ω-3 PUFAs and another drug in combination, the dose of ω-3 PUFAs and/or another drug may be set lower than a conventional dose for general use. For instance, each drug may be used at a dose inadequate to gain therapeutic effects from the relevant drug alone. In that case, side effects of drug administration are reduced with advantage.

If the dose of ω-3 PUFAs and/or another drug is inadequate to gain therapeutic effects from the relevant drug alone, it is also desirable that the therapeutic effects of the ω-3 PUFAs and another drug as applied in combination exceed the total effects of the ω-3 PUFAs and another drug as applied separately at the same doses as those upon the application in combination.

The dose of ω-3 PUFAs which is inadequate to gain therapeutic effects from them alone, as varying with the condition or habitus of each individual subject, is not limited, and is exemplified by the daily dose of EPA-E and/or DHA-E which is not less than 0.1 g but less than 2 g, and is preferably 0.2 to 1.8 g, more preferably 0.3 to 0.9 g, especially 0.3 to 0.6 g.

With respect to another drug, as well as ω-3 PUFAs, the daily dose, the frequency of administration, or the dosage ratio is not particularly limited but can be modified appropriately by examining test values concerning the degree of liver fibrosis, the reduction in serum AST or ALT level, the reduction in AST/ALT ratio, the increase in adiponectin, the reduction in TNFα or reduction in oxidative stress marker in blood, the improvement in HOMA-IR, and so forth.

The active ingredient or ingredients of the pharmaceutical composition of the present invention may be administered as a compound (optionally including other constituents unremovable by purification) in itself, or combined with excipients suitably selected from among conventional carriers or media, vehicles, binders, lubricants, colorants, flavors, sterilized water or vegetable oils as required, as well as innoxious organic solvents or innoxious solubilizing agents (e.g., glycerin, propylene glycol), emulsifiers, suspending agents (e.g., Tween 80, gum arabic solution), isotonicities, pH-adjusting agents, stabilizers, soothing agents, corrigents, flavoring agents, preservatives, antioxidants, buffers, colorants, and the like, so as to prepare an appropriate medical formulation. Exemplary excipients which may be contained include lactose, partially pregelatinized starch, hydroxypropylcellulose, macrogol, tocopherol, a hydrogenated oil, a sucrose ester of fatty acid, hydroxypropylmethylcellulose, titanium oxide, talc, dimethylpolysiloxane, silicon dioxide, and carnauba wax.

Since ω-3 PUFAs are of a highly unsaturated nature, it is particularly desirable to add an effective amount of an antioxidant, for instance, at least one selected from among butylated hydroxytoluene, butylated hydroxyanisole, propyl gallate, gallic acid, an pharmaceutically acceptable quinone, and α-tocopherol.

The dosage form of the formulation, as varying with the mode of combined application of active ingredients according to the present invention, is not particularly limited. The formulation may be administered to a subject orally, intravenously, intraarterially, by inhalation, rectally, intravaginally or externally, that is to say, as an oral formulation in the form of tablet, film-coated tablet, capsule, microcapsule, granule, fine granule, powder, oral liquid preparation, syrup, jelly, inhalant or the like, or as a parenteral formulation in the form of ointment, suppository, injection (emulsion, suspension, nonaqueous solution), solid injection to be emulsified or suspended before use, transfusion solution, external preparation such as endermic preparation, or the like. For those subjects who are able to take oral formulations, easy-to-take oral formulations are desirable, so that oral administration of the formulation as included in a capsule such as soft capsule and microcapsule, in tablet form, or in film-coated tablet form is particularly preferred. It is also possible to administrate the formulation orally as an enteric preparation or an extended release preparation, or as a jelly in the case of dialysis patients or patients with dysphagia.

If two formulations prepared from ω-3 PUFAs and another drug, respectively, are combined with each other for use as the composition of the present invention, the formulations are each prepared by a known method. The composition of the invention may also be prepared as a composite formulation containing ω-3 PUFAs and another drug as active ingredients.

If the pharmaceutical composition of the present invention is to be prepared as a composite formulation containing ω-3 PUFAs and another drug as active ingredients, the composite formulation is not particularly limited in dosage form, so that it is administered to a subject as an oral formulation in the form of tablet, film-coated tablet, capsule, microcapsule, granule, fine granule, powder, oral liquid preparation, syrup, jelly or the like, or as a parenteral formulation in the form of injection, transfusion solution, external preparation such as endermic preparation, or the like. In addition, the composite formulation includes a formulation made adapted for extended release, a formulation releasing two drugs separately with a certain time lag, and so forth.

The composite formulation of the present invention may contain a pharmaceutically acceptable vehicle in addition to active ingredients. The formulation may also contain a known antioxidant, coating agent, gelling agent, corrigent, flavoring agent, preservative, antioxidant, emulsifier, pH-adjusting agent, buffer, colorant or the like as appropriate.

The composite formulation of the present invention can be prepared by any method practicable by a person of ordinary skill in the art of the present invention. Preferably, the composite formulation can be prepared according to a usual manner. Powder of ω-3 PUFAs is obtained by a known method in which, for instance, an oil-in-water emulsion containing (A) EPA-E, (B) dietary fiber, (C) a starch hydrolysate and/or a reducing starch decomposition product obtained by saccharification into oligosaccharide, and (D) a water-soluble antioxidant is dried in a high vacuum, then pulverized (JP 10-99046 A). By using the powder of EPA-E thus obtained and powder of a biguanide-type hypoglycemic agent, a formulation in the form of granule, fine granule, powder, tablet, film-coated tablet, chewable tablet, extended release tablet, orally-disintegrating tablet (OD tablet) or the like can be prepared preferably according to a usual manner. Chewable tablets may be obtained by the known method in which EPA-E is emulsified in a solution of water-soluble polymer such as hydroxypropylmethylcellulose, and the resultant emulsion is sprayed onto lactose or other excipient to form powdery glanules (JP 8-157362 A), with the granules being mixed with the powder of a biguanide-type hypoglycemic agent for compressing. Extended release tablets may be obtained by (1) forming two layers containing EPA-E and a biguanide-type hypoglycemic agent, respectively, so as to arrange one layer inside and the other outside, or (2) forming two matrix disks containing the two ingredients, respectively, so as to layer them, or (3) embedding particulate capsules including one ingredient into a matrix containing the other ingredient, or (4) mixing the two drugs together, then subjecting the mixture to some measures for extended release. It is desirable that the active ingredients are each regulated in releasing rate, and the two drugs may be released simultaneously or separately with a certain time lag. Orally-disintegrating tablets may be produced in accordance with such a known method as disclosed in JP 8-333243 A, and a film preparation for oral cavity may be produced in accordance with such a known method as disclosed in JP 2005-21124 A.

It is desirable that the active ingredients of the composite formulation of the present invention are so released and absorbed that their pharmacological actions may be exerted. Preferably, the composite formulation of the present invention has at least one effect out of an improved active-ingredient release, enhancement of the absorbability of active ingredients, enhancement of the dispersibility of active ingredients, an improved storage stability of the formulation in itself, and enhancement of the convenience to subjects taking the formulation, or improvement of the compliance of such patients.

The composition of the present invention is effective at preventing/ameliorating or treating NAFLD, NASH in particular, preventing recurrence thereof, or suppressing progression thereof to cirrhosis or liver cancer in an animal, especially mammal. Exemplary mammals include humans, livestock animals such as cows, horses and pigs, as well as domestic animals such as dogs, cats, rabbits, rats and mice, with humans being preferred subjects. The composition of the present invention is particularly suitable for patients with NASH having diabetes, hyperlipidemia, hypertriglyceridemia, metabolic syndrome, hypertension, or insulin resistance. The inventive composition is also effective for those subjects having high uric acid levels, suffering from a severer systemic inflammation, taking multiple drugs other than ω-3 PUFAs, or having experienced side effects of a taken drug other than ω-3 PUFAs. In addition, the composition of the present invention as provided in the form of a composite formulation or a formulation kit relieves the burden of medication on a subject to improve the medication compliance of the subject, leading to further enhanced prophylactic/ameliorative or therapeutic effects.

The following are examples of the present invention, to which the present invention is in no way limited.

### Example 1

Subjects affirmatively diagnosed as having NASH are divided into two groups (each comprising 15 cases), namely the EPA-E group and the control group, then the groups are caused to take EPADEL S900 (containing 900 mg of EPA-E) and a placebo, respectively, twice a day. Doses are modified as appropriate to the subjects' conditions. Following the method described in American Journal of Gastroenterology 2001, Vol. 96, pp. 2711-2717 with respect to the criteria for diagnosing subjects, monitoring, histological testing, statistic analysis, and so forth, blood chemistry tests on ALT, AST, and the like are performed and the plasma fatty acid composition is determined over a dosage period of one year, and liver biopsy is conducted at the end of dosage, so as to make histological evaluations.

The mean values of blood chemistry parameters, such as ALT and AST, of the EPA-E group are reduced from those before treatment more markedly in comparison with the control group. In the EPA-E group, amelioration of the average fibrosis from stage 2 to stage 1 according to the fibrosis staging of Brunt et al. is observed on the liver tissue images under pathological examination.

Of the EPA-E group, 15 cases are sorted into two groups, one (“effective” group) comprising the cases in which the score in accordance with the method of Brunt et al. is changed after the administration of the drug from that before the administration by 1 or 2, and the other (“ineffective” group) comprising the cases in which the score after the administration is not changed from that before the administration, and analysis is conducted on the change in the plasma fatty acid composition as determined over the dosage period of one year. The OA/SA ratio before the administration is not different between the effective group and the ineffective group. After the start of the administration, the OA/SA ratio tends to be reduced in both groups for two months, while the reduction rate of the OA/SA ratio in two months after the start of the administration is high in the effective group as compared with the ineffective group. The reduction rate of the OA/SA ratio in two months is calculated by the equation: reduction rate (%)=[(OA/SA ratio before start of administration)−(OA/SA ratio two months after start of administration)]/OA/SA ratio before start of administration×100. Two months after the start of the administration, the OA/SA ratio in the effective group is lower in value than in the ineffective group. In both of the effective and ineffective groups, the OA/SA ratio from the lapse of two months on tends more or less to be reduced, although at a low rate as compared with the ratio for two months after the start of the administration. In the effective group, hyaluronic acid, type IV collagen, and TIMP-1 are gradually reduced in value. In contrast, hyaluronic acid, type IV collagen, and TIMP-1 in the ineffective group are hardly changed in value.

### Example 2

Subjects affirmatively diagnosed as having NASH are divided into two groups, one (group with OA/SA ratio increase) comprising the subjects in whom the OA/SA ratio is increased as compared with that determined within the preceding one year, and the other (group without OA/SA ratio increase) comprising the rest, then each group is caused to take EPADEL S900 (containing 900 mg of EPA-E) twice a day. Doses are modified as appropriate to the subjects' conditions. Following the method described in American Journal of Gastroenterology 2001, Vol. 96, pp. 2711-2717 with respect to the criteria for diagnosing subjects, monitoring, histological testing, statistic analysis, and so forth, blood chemistry tests on ALT, AST, and the like are performed and the plasma fatty acid composition is determined over a dosage period of one year, and liver biopsy is conducted at the end of dosage, so as to make histological evaluations. Therapeutic effects are evaluated by the NAS scoring of Kleiner et al.

In both the group with OA/SA ratio increase and the group without OA/SA ratio increase, NAS scores are improved owing to the administration of the drug for one year. At the same time, the degree of improvement in NAS score is higher in the group with OA/SA ratio increase than in the group without OA/SA ratio increase.

### Example 3

Patients with NASH having liver fibrosis, that is to say, subjects suffering from fibrosis classified to stage 1 or 2 according to the fibrosis staging of Brunt et al. are collected (15 cases). Dietary restriction and exercise therapy are conducted so that the blood OA/SA ratio as monthly measured may be reduced in value from that measured previously, while 900 mg of EPA-E is administered twice a day, so as to do follow-up for one year. To the subjects in whom no change is recognized in OA/SA ratio, 900 mg of EPA-E is administered thrice a day.

The subjects in whom the reduction ratio of the OA/SA ratio in one month after the start of the administration is not lower than 5% are included in group A, while the subjects in whom the reduction ratio of the OA/SA ratio in one month after the start of the administration is lower than 1% are included in group B. The reduction rate of the OA/SA ratio in one month is calculated by the equation: reduction rate (%)=[(OA/SA ratio before start of administration)−(OA/SA ratio one month after start of administration)]/OA/SA ratio before start of administration×100.

It is observed by liver biopsy conducted one year after the start of treatment that fibrosis in the subjects of group A is ameliorated from stage 2 to stage 1 or non-fibrotic stage, or again, from stage 1 to non-fibrotic stage. Amelioration of fibrosis in group B is slight, that is to say, not so significant as in group A.

### Example 4

There are three genotypes for the adiponectin gene SNP276, the T/T, G/T and G/G genotypes, and humans with the G/G genotype are said to have a lower blood adiponectin level than those with the T/T genotype.

To a group of patients with NASH having the adiponectin gene SNP276 which is of the G/G genotype and a group of patients with NASH having the adiponectin gene SNP276 which is of the genotype other than the G/G genotype (each group comprising 15 cases), 900 mg of EPA-E is administered twice a day for one year. After the lapse of one year, therapeutic effects are evaluated by the NAS scoring of Kleiner et al. In both of the group of patients with NASH having the G/G genotype and the group of patients with NASH having the genotype other than the G/G genotype, NAS scores are improved owing to the administration of the drug for one year. At the same time, the degree of improvement in NAS score is higher in the group of patients with NASH having the G/G genotype than in the group of patients with NASH having the genotype other than the G/G genotype.

### Example 5

A subject affirmatively diagnosed as having NASH is treated by using the treatment method of the present invention.

Before the start of treatment, the plasma fatty acid composition of the subject is determined, and the OA/SA ratio is calculated, with a value of 3.5 being obtained. The subject is caused to take EPADEL S900 (containing 900 mg of EPA-E) twice a day. After the lapse of one month, the OA/SA ratio of the subject remains 3.5. The subject is instructed on diet, whereupon an exercise therapy is additionally conducted. Three months after the start of the administration of the drug, the OA/SA ratio of the subject is reduced to 3.4. EPADEL S900 (containing 900 mg of EPA-E) is administered thrice a day to increase the daily dose. Six months after the start of the administration of the drug, the OA/SA ratio of the subject is reduced to 3.2. Thereafter, the dietary restriction and the exercise therapy, and a thrice-a-day administration of EPADEL S900 (containing 900 mg of EPA-E) as well, are continued. An OA/SA ratio of about 3.2 or 3.1 is maintained.

Blood chemistry tests on ALT, AST, and the like are performed and the plasma fatty acid composition is determined over a dosage period of one year, and liver biopsy is conducted at the end of dosage, so as to make histological evaluations. Amelioration of the average fibrosis is observed, from stage 2 to stage 1 according to the fibrosis staging of Brunt et al. In addition, ALT, AST, hyaluronic acid, type IV collagen, and TIMP-1 are kept reduced in value for a period of one year.

### Example 6

30 Subjects diagnosed by liver biopsy as having NASH are divided into two groups, namely the treatment group comprising 20 cases and the control group comprising 10 cases, then 2700 mg/day of high purity EPA-E (at least 96.5% by weight pure; trade name, EPADEL S) is administered to the treatment group, and a placebo to the control group, each for 12 months.

Various tests are conducted on the subjects before the start of the administration, as well as one month, three months, six months and 12 months after the start of the administration, so as to evaluate therapeutic effects.

Test items are selected as appropriate from among plasma total fatty acid, plasma free fatty acid, liver ultrasonography, body weight, ALT, AST, HbAlc, glucose level, HOMA-IR, TNFα, sTNF-R1, R2, ferritin, thioredoxin, TGF-β1, TIMP-1, hyaluronic acid, and so forth. Therapeutic effects on the subjects are evaluated 12 months after the start of the administration by the NAS scoring.

The average degree of improvement in NAS score 12 months after the start of the administration is high in the treatment group as compared with the control group. ω-3 PUFAs are useful as a therapeutic agent against NASH.

In 20 cases of the treatment group, the relationship between the degree of improvement in NAS score and the change in blood fatty acid ratio in one month after the start of the administration of the drug is reviewed. The change in blood fatty acid ratio in one month after the start of the administration is determined by measuring blood fatty aced levels before the start of the administration of ω-3 PUFAs and one month after the start of the administration, calculating one or more out of:

(1) the oleic acid/stearic acid ratio,
(2) the stearic acid/palmitic acid ratio,
(3) the oleic acid/palmitic acid ratio, and
(4) palmitoleic acid/palmitic acid ratio, and comparing in value the ratio or ratios before the start of the administration with those one month after the start of the administration.

The blood fatty acid level ratios as above are reduced one month after the start of the administration as compared with those before the start of the administration in subjects with improved NAS scores, that is to say, the subjects in whom therapeutic effects on NASH are obtained by the administration of ω-3 PUFAs.

Therapeutic effects on NASH are achieved by the administration of ω-3 PUFAs particularly in the subjects whose oleic acid/stearic acid ratio is reduced in value one month after the start of the administration as compared with that before the start of the administration. By continuing administering ω-3 PUFAs to such subjects, NASH can be treated.

The subjects with higher degrees of improvement in NAS score, that is to say, the subjects in whom the therapeutic effects on NASH of the administration of ω-3 PUFAs are more significant tend to have higher reduction rates of the blood fatty acid ratios as above in one month after the start of the administration. Whether or not therapeutic effects are gained from ω-3 PUFAs is predictable by measuring fatty acids in blood before the start of the administration and one month after the start of the administration, and obtaining the reduction rate of a specified blood fatty acid ratio.

The fatty acids in blood to be employed for the calculation of the blood fatty acid ratios as above may each be a plasma total fatty acid or a plasma free fatty acid. The reduction rate of a blood fatty acid ratio may be obtained not only from the variation in a certain period of time after the start of administration of a drug but the variation in a certain period of time within the dosage period.

The therapeutic effects on NASH of the administration of ω-3 PUFAs may also be evaluated totally on the basis of a suitable combination of the variation in blood fatty acid ratio with TNFα, ferritin, thioredoxin, TIMP-1, TGF-β1, and so forth.

1. (canceled)

2. A method of preventing/ameliorating or treating non-alcoholic steatohepatitis, which comprises using values of one or more selected from the group consisting of a plasma oleic acid/stearic acid ratio, a plasma stearic acid/palmitic acid ratio and a plasma oleic acid/palmitic acid ratio of a subject as indices for evaluation of the subject's condition or therapeutic effects, and administering to the subject a pharmaceutical composition comprising as an active ingredient at least one selected from the group consisting of ω-3 polyunsaturated fatty acids as well as pharmaceutically acceptable salts and esters thereof.

3. (canceled)

4. A method of preventing/ameliorating or treating non-alcoholic steatohepatitis in a subject suffering from non-alcoholic steatohepatitis, comprising the steps of:
(1) obtaining a first determination with respect to at least one out of a plasma oleic acid/stearic acid ratio, a plasma stearic acid/palmitic acid ratio and a plasma oleic acid/palmitic acid ratio of the subject;
(2) administering to the subject a pharmaceutical composition comprising as an active ingredient at least one selected from the group consisting of ω-3 polyunsaturated fatty acids as well as pharmaceutically acceptable salts and esters thereof;
(3) obtaining a second determination with respect to at least one out of the plasma oleic acid/stearic acid ratio, the plasma stearic acid/palmitic acid ratio and the plasma oleic acid/palmitic acid ratio of the subject;
(4) making a comparison between the first and second determinations in order to evaluate the subject's condition;
(5) evaluating, based on the comparison between the first and second determinations, a treatment given to the subject in order to specify an appropriate therapeutic dosage of the pharmaceutical composition, the composition being suitable for prevention/amelioration or treatment of non-alcoholic steatohepatitis; and
(6) thus preventing/ameliorating or treating non-alcoholic steatohepatitis.

5. (canceled)

6. The method according to claim 4, wherein the step of making a comparison between the first and second determinations further includes determining upon making the comparison whether or not at least one out of the plasma oleic acid/stearic acid ratio, the plasma stearic acid/palmitic acid ratio and the plasma oleic acid/palmitic acid ratio of the subject is reduced.

7. The method according to claim 4, wherein the pharmaceutical composition is administered for one month before the second determination is obtained.

8. The method according to claim 6, wherein the pharmaceutical composition is administered for one month before the second determination is obtained.
